(19) United States
(12) Patent Application Publication
(10) Pub. No.: US 2005/0182384 A1
(43) Pub. Date: Aug. 18, 2005

(54) Flushing cannula with integral catheter sheath

(75) Inventor: John D. Kantor, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/931,542

(22) Filed: Aug. 31, 2004

Related U.S. Application Data
(60) Provisional application No. 60/543,881, filed on Feb. 12, 2004.

(51) Int. Cl. A61M 31/00; A61F 2/06

(52) U.S. Cl. 604/500; 623/1.42

(57) ABSTRACT

The invention provides a catheter flushing device. The device comprises a syringe fitting, a hollow stylet, and a sleeve. The hollow stylet is attached to and in fluid communication with the syringe fitting. The sleeve is attached to the syringe fitting external to and coaxial with the hollow stylet. The hollow stylet is received within a distal end of a catheter, the sleeve surrounding a distal portion of the catheter. The syringe fitting and hollow stylet serve as a flushing cannula for the catheter. The sleeve surrounds a distal portion of the catheter, protecting a treatment device carried on the catheter from contact with the flushing solution. The flushing device is attached to a catheter prior to shipment by the catheter manufacturer or supplier, thus saving the end user time when preparing the catheter for use, and also protecting the treatment device during shipping and storage.

### RELATED APPLICATIONS

This application claims the benefit of U.S. Provisional Patent Application 60/543,881 filed Feb. 12, 2004.

### TECHNICAL FIELD

This invention relates generally to biomedical devices that are used for treating vascular conditions. More specifically, the invention relates to a device that combines a flushing cannula with a catheter sheath to protect a treatment device carried on a catheter during flushing of the catheter with a saline or other solution and also to protect the treatment device during shipping and storage of the catheter.

### BACKGROUND OF THE INVENTION

Heart disease, specifically coronary artery disease, is a major cause of death, disability, and healthcare expense in the United States and other industrialized countries. In atherosclerosis, one form of heart disease, deposits of hard plaques (atheromas) are formed within the inner coat of a vessel (intima) and inner media of arteries. This atherosclerotic disease process leads to a critical narrowing (stenosis) of the affected coronary artery and produces anginal syndromes, known commonly as chest pain.

One method for treating atherosclerosis and other forms of vascular narrowing is percutaneous transluminal coronary angioplasty (PTCA). During PTCA, a balloon catheter device is inflated within the stenotic vessel. Upon inflation, the pressurized balloon exerts a compressive force on the lesion, thereby increasing the inner diameter of the affected vessel.

Soon after the procedure, however, a significant proportion of treated vessels restenose. To prevent restenosis, a stent may be implanted within the vessel. The stent acts as a scaffold to support the lumen in an open position and maintain lumen size. For insertion, the stent is affixed in a compressed configuration along the delivery catheter, for example crimped onto a balloon that is folded or otherwise wrapped about a guidewire. After the stent is properly positioned within the vessel, it is expanded, causing its diameter to expand.

Because stent insertion can cause undesirable reactions such as inflammation, infection, thrombosis, or proliferation of cell growth that occludes the passageway, stents are sometimes coated with therapeutic agents to assist in preventing these conditions. The coatings are bioengineered to release precise doses of the therapeutic agent. However, if the coating remains in direct contact with another material for an extended period of time, for example during shipping and storage, components of the therapeutic agent may migrate into the other material, resulting in delivery of a lower dose of the therapeutic agent than intended. Alternatively, components of a packaging material may migrate into the therapeutic coating, again leading to impaired performance.

A packaging sheath such as that disclosed in U.S. Pat. No. 6,030,407 to Eidenschink minimizes contact of the stent with the packaging material and so protects the stent during shipping and storage. However, this type of sheath must be removed before the catheter can be prepared for use. A catheter is commonly flushed with a saline solution immediately prior to use to confirm patency of the catheter and to increase lubricity of the lumen of the catheter, for example to aid in passing the catheter over a guidewire. A coated stent may be damaged if the saline solution is inadvertently flushed not only through the lumen of the catheter but also over the stent.

A flushing cannula is commonly provided by the catheter manufacturer to direct the saline solution into the lumen of the catheter. However, inserting the cannula adds to the time required for a procedure, and so not all end users choose to employ the cannula. Even when a flushing cannula is used, if fluid escapes from the cannula, an unsheathed coated stent is at risk of being damaged by the fluid.

Therefore, it would be desirable to have an improved catheter flushing device that overcomes the aforementioned and other disadvantages.

### SUMMARY OF THE INVENTION

One aspect of the present invention is a catheter flushing device. The device comprises a syringe fitting, a hollow stylet, and a sleeve. The hollow stylet is attached to a proximal end of the syringe fitting, the hollow stylet in fluid communication with the syringe fitting. The sleeve is attached to an outer surface of the syringe fitting, external to and coaxial with the hollow stylet.

Another aspect of the present invention is a catheter system. The system comprises a catheter, a treatment device carried on a distal portion of the catheter, a syringe fitting, a hollow stylet, and a sleeve. The hollow stylet is attached to a proximal end of the syringe fitting such that the hollow stylet is in fluid communication with the syringe fitting. The sleeve is attached to an outer surface of the syringe fitting, external to and coaxial with the hollow stylet. The hollow stylet is received within a distal end of the catheter. The syringe fitting and hollow stylet serve as a flushing cannula for the catheter. The sleeve surrounds a distal portion of the catheter, protecting it from contact with the flushing solution.

Yet, another aspect of the present invention is a method of preparing a catheter for use. A catheter system is provided, the system comprising a catheter and a catheter flushing device. The catheter flushing device includes a syringe fitting, a hollow stylet, and a sleeve. The hollow stylet is attached to and in fluid communication with the syringe fitting. The sleeve is attached to the syringe fitting external to and coaxial with the hollow stylet. The hollow stylet is received within a distal end of the catheter, the sleeve surrounding a distal portion of the catheter. The syringe fitting and hollow stylet serve as a flushing cannula for the catheter. The sleeve surrounds a distal portion of the catheter, protecting it from contact with the flushing solution. A syringe containing a flushing solution is attached to the syringe fitting. The flushing solution is injected into the catheter. The catheter flushing device is removed from the catheter.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a catheter flushing device, in accordance with the present invention;

FIG. 2 is an illustration of one embodiment of a catheter system, in accordance with the present invention;

FIG. 3 is an illustration of another embodiment of a catheter system, in accordance with the present invention; and

FIG. 4 is a flow diagram of one embodiment of a method of preparing a catheter for use, in accordance with the present invention.

### DETAILED DESCRIPTION OF THE PRESENTLY PREFERRED EMBODIMENTS

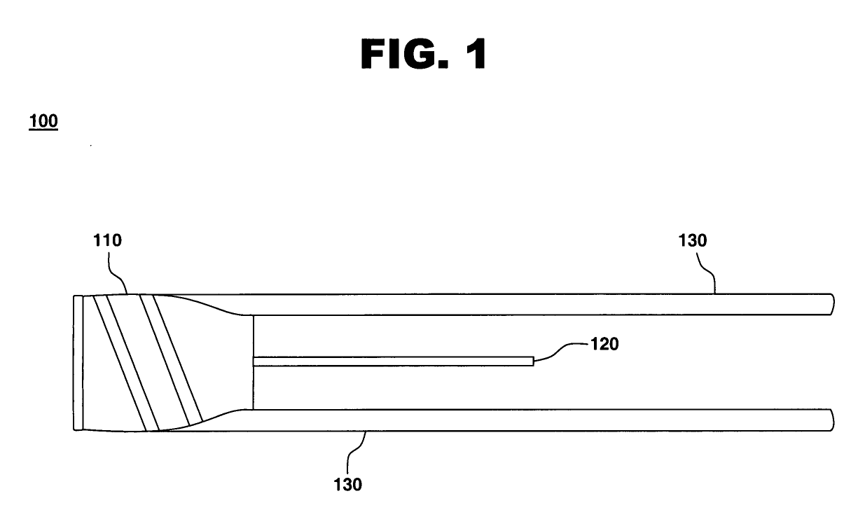

One aspect of the present invention is a catheter flushing device. One embodiment of the device, in accordance with the present invention, is illustrated in FIG. 1 at 100. The device comprises a syringe fitting 110, a hollow stylet 120, and a sleeve 130.

In the present example, syringe fitting 110 is a standard Luer fitting. One skilled in the art will recognize that any appropriate syringe fitting may be used; however, the Luer fitting is the most common syringe fitting and, therefore, preferred. Syringe fitting 110 may be made of a typical Luer fitting material such as a polycarbonate or polyethylene.

Hollow stylet 120 is attached to a proximal end of syringe fitting 110 and is in fluid communication with the syringe fitting. Thus, a syringe connected to syringe fitting 110 may inject a saline solution, for example, through both the fitting and the hollow stylet. In the present example, stylet 120 is a stainless steel hypotube, but other appropriate materials may be used, including but not limited to nitinol, cobalt alloys, titanium, or any biocompatible metal. Stylet 120 may be attached to syringe fitting 110 using an adhesive or by another appropriate method known in the art.

Sleeve 130 is attached to an outer surface of syringe fitting 110. As can be seen in FIG. 1, the sleeve is external to and coaxial with hollow stylet 120. The sleeve may extend beyond the stylet, as in FIG. 1, or the stylet may extend outside the sleeve. Sleeve 130 may be fabricated using the same material as syringe fitting 110, or it may comprise a different material that is bondable to the syringe fitting material. Where the catheter flushing device is to be used with a catheter bearing a coated stent, sleeve 130 preferably comprises one or more materials nonreactive with a coated stent, for example polypropylene, polyethylene, a nylon/polyethylene blend, PTFE (polytetrafluoroethylene), a suitable polycarbonate, or any biocompatible or coated-stent-compatible material. Sleeve 130 may be attached to syringe fitting 120 using a method such as laser welding, RF (radio frequency) welding, heat bonding, convection heating, or any appropriate bonding method.

In another example of the present embodiment, syringe fitting 110, hollow stylet 120, and sleeve 130 may be fabricated as a unitary structure. A molding method such as blow molding or injection molding may be used to form the elements of the device in a single process. As noted above, where the device is to be used to flush a catheter bearing a coated stent, the entire device is preferably fabricated using one or more nonreactive materials, for example polypropylene, polyethylene, a nylon/polyethylene blend, PTFE, or a suitable polycarbonate. Alternatively, syringe fitting 110 and sleeve 130 may be formed as a unitary structure, and hollow stylet 120 may be attached thereto.

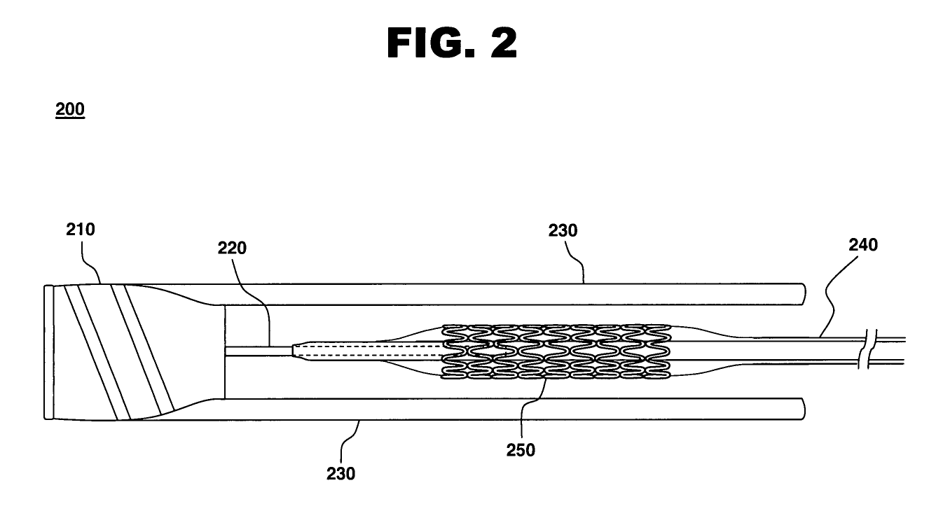

Another aspect of the present invention is a catheter system. One embodiment of the system, in accordance with the present invention, is illustrated in FIG. 2 at 200. System 200 comprises a syringe fitting 210, a hollow stylet 220, a sleeve 230, a catheter 240, and a treatment device, in this embodiment a coated stent 250.

Syringe fitting 210 is shown in FIG. 2 as a Luer fitting; however, any appropriate syringe fitting may be used. Hollow stylet 220 is attached to a proximal end of syringe fitting 210 and is in fluid communication with the syringe fitting. Sleeve 230 is attached to an outer surface of syringe fitting 210 such that the sleeve is external to and coaxial with hollow stylet 220.

Hollow stylet 220 is received within a distal end of catheter 240, typically within an inner member of the catheter. Syringe fitting 210 and hollow stylet 220 serve as a flushing cannula for catheter 240. Thus, a syringe connected to syringe fitting 210 can inject a flushing solution into catheter 240 through the fitting and the hollow stylet to flush the catheter immediately prior to use.

Hollow stylet 220 may be sized such that it is retained within the distal end of catheter 240 by friction. When hollow stylet 220 is in place within catheter 240, sleeve 230 surrounds a distal portion of the catheter, protecting it from contact with a flushing solution. In the present example, the distal portion of catheter 240 carries coated stent 250. In another example, catheter 240 may carry a treatment device such as an angioplasty balloon or an electronic device.

Stent 250 may be coated with an antineoplastic agent, an antiproliferative agent, an antibiotic, and antithrombogenic agent, and anticoagulant, an antiplatelet agent, an anti-inflammatory agent, or a combination thereof. To protect against components of the therapeutic agent migrating into the sleeve material or components of the sleeve material migrating into the therapeutic coating, sleeve 230 may be sized such that it does not contact the coated stent. To provide further protection, sleeve 230 may be fabricated using a nonreactive material such as polypropylene, polyethylene, a nylon/polyethylene blend, PTFE, a suitable polycarbonate, or any biocompatible or coated-stent-compatible material. A treatment device other than a coated stent may be better protected by a sleeve that contacts the device, in which case a highly flexible or elastomeric sleeve material may be chosen.

Syringe fitting 210, hollow stylet 220, and sleeve 230 may be fabricated as a unitary structure, for example using a molding method. Alternatively, the components may be manufactured separately and assembled. For example hollow stylet 220 may be a stainless steel hypotube that is attached to syringe fitting 210 using an adhesive, and a separately manufactured sleeve 230 may be bonded to syringe fitting 220. In yet another alternative, syringe fitting 210 and sleeve 230 may be fabricated as a unitary structure, with hollow stylet 220 formed separately and attached to this structure. Hollow stylet 220 may be formed using materials other than stainless steel, including but not limited to nitinol, cobalt alloys, titanium, or any biocompatible metal.

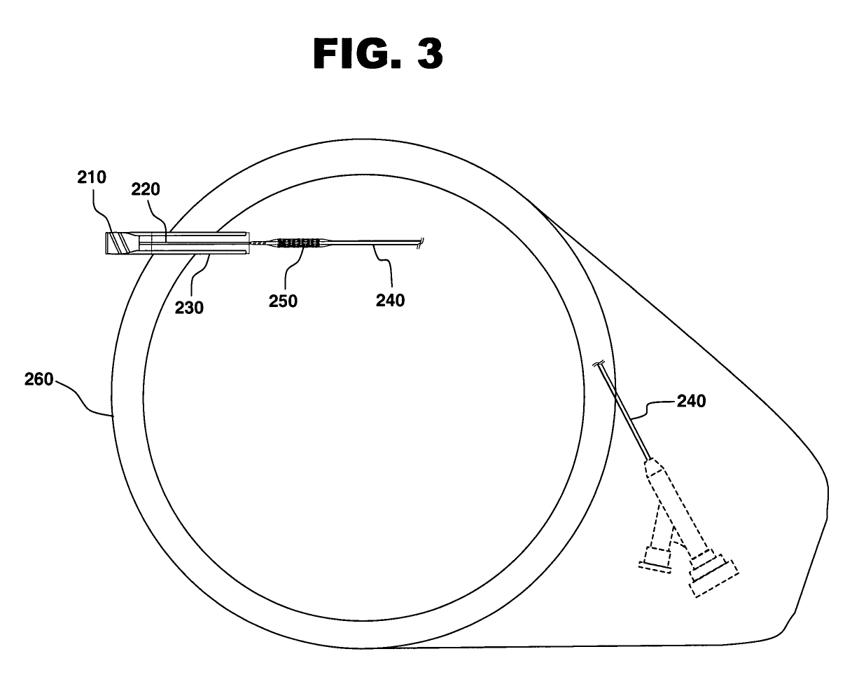

A catheter system in accordance with the present invention may further comprise a shipping and storage container 260, as illustrated in FIG. 3. Like elements share like reference numbers in FIG. 2 and FIG. 3.

Syringe fitting 210 extends through a wall of container 260, with one or both of syringe fitting 210 and sleeve 230 forming a unitary structure with the wall. For example, syringe fitting 210, sleeve 230, and container 260 may be formed in a single molding process, with hollow stylet 220 formed separately and attached to a proximal end of syringe fitting 210 using an adhesive. Alternatively, hollow stylet 220 may be formed integral with syringe fitting 210, sleeve 230, and container 260. Thus, during packaging, catheter 240 may be loaded onto hollow stylet 220 such that the stylet is received within a distal end of the catheter, with sleeve 230 surrounding a distal portion of the catheter. Stylet 220 may extend outside of sleeve 230, as shown in FIG. 3, for ease of loading the catheter onto the stylet. Alternatively, hollow stylet 220 may be formed as a separate member. A proximal portion of hollow stylet 220 may be received within the distal end of catheter 240, and the distal end of hollow stylet 220 may then be snapped or otherwise assembled into the proximal end of syringe fitting 210.

In another example, syringe fitting 210, hollow stylet 220, and sleeve 230 may be separate from shipping and storage container 260. In this example, catheter 240 is loaded onto hollow stylet 220 with sleeve 230 surrounding a distal portion of the catheter, and the resulting assembly is then placed into shipping and storage container 260. The shipping and storage container is designed to permit syringe fitting 210 to extend out through an opening in the wall of container 260. The system would appear as in FIG. 3, but the syringe fitting and sleeve would seat into the container rather than being part of it.

In the described examples, the proximal portion of catheter 240 may be coiled within the shipping and storage container, with the proximal end of the catheter either inside or outside the shipping container. Catheter 240 may be flushed while still held within container 260, with the flushing solution either captured within the container or expelled outside the container. Sleeve 230 protects the distal portion of catheter 240, which is here shown bearing a coated stent 250, from contact with the flushing solution. A cap (not shown) may be removably attached to syringe fitting 210, if desired, to protect or seal the fitting.

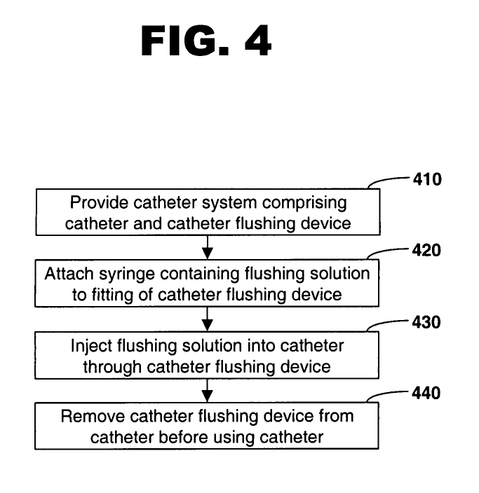

Yet another aspect of the present invention is a method of preparing a catheter for use. FIG. 4 shows a flow diagram of one embodiment of the method, in accordance with the present invention.

A catheter system is provided (Block 410). The catheter system comprises a catheter and a catheter flushing device. The catheter flushing device includes a syringe fitting, a hollow stylet, and a sleeve. The hollow stylet is attached to and in fluid communication with the syringe fitting. The sleeve is attached to the syringe fitting external to and coaxial with the hollow stylet. The hollow stylet is received within a distal end of the catheter, the sleeve surrounding a distal portion of the catheter. The catheter distal portion may carry a treatment device such as a coated stent. The catheter system may further comprise a shipping and storage container designed such that the syringe fitting extends through a wall of the container.

The syringe fitting and hollow stylet serve as a flushing cannula for the catheter. The described catheter system is assembled by the catheter manufacturer or supplier, thus providing the end user with a flushing cannula already installed in the catheter, saving the end user time when preparing the catheter for use. The sleeve not only protects a treatment device from contact with a flushing solution during preparation of the catheter, but also protects the treatment device from damage during shipping and storage.

A syringe containing a flushing solution is attached to the syringe fitting (Block 420). The syringe fitting may be a standard Luer fitting, in which case attaching the syringe to the syringe fitting comprises rotating the syringe onto the Luer fitting.

The flushing solution is injected into the catheter (Block 430). The sleeve of the catheter flushing device protects a treatment device carried on the catheter from coming into contact with the flushing solution. Where the catheter system includes a shipping and storage container, the catheter may be flushed while still held within the container, with the flushing solution either captured within the container or expelled outside the container.

The catheter flushing device is removed from the catheter prior to use (Block 440). Where the catheter flushing device is independent of a shipping and storage container, this may be accomplished by simply pulling the catheter flushing device out of and away from the distal end of the catheter. Where the catheter flushing device is an integral part of a shipping and storage container, the catheter flushing device is removed by withdrawing the distal portion of the catheter from the device and removing the remaining portion of the catheter from the container.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

1. A catheter flushing device, comprising:
a syringe fitting;
a hollow stylet attached to a proximal end of the syringe fitting, the hollow stylet in fluid communication with the syringe fitting; and
a sleeve attached to an outer surface of the syringe fitting, the sleeve external to and coaxial with the hollow stylet.

2. The device of claim 1 wherein the syringe fitting is a Luer fitting.

3. The device of claim 1 wherein the hollow stylet is a stainless steel hypotube.

4. The device of claim 1 wherein the hollow stylet is attached to the syringe fitting using an adhesive.

5. The device of claim 1 wherein the sleeve is fabricated using one or more materials selected from a group consisting of polypropylene, polyethylene, a nylon/polyethylene blend, polytetrafluoroethylene (PTFE), and a suitable polycarbonate.

6. The device of claim 1 wherein the sleeve is attached to the syringe fitting using a method selected from a group consisting of laser welding, RF welding, heat bonding, convection heating, and an appropriate bonding method.

7. The device of claim 1 wherein the syringe fitting, hollow stylet, and sleeve are fabricated as a unitary structure.

8. The device of claim 7 wherein the unitary structure is fabricated using one or more methods selected from a group consisting of blow molding, injection molding, and a suitable molding method.

9. A catheter system, comprising:
a treatment device;
a catheter, a distal portion thereof carrying the treatment device;
a syringe fitting;
a hollow stylet attached to a proximal end of the syringe fitting such that the hollow stylet is in fluid communication with the syringe fitting; and
a sleeve attached to an outer surface of the syringe fitting such that the sleeve is external to and coaxial with the hollow stylet, wherein the hollow stylet is received within a distal end of the catheter, the sleeve surrounding the distal portion of the catheter, the syringe fitting and hollow stylet serving as a flushing cannula for the catheter, the sleeve protecting the distal portion of the catheter from contact with a flushing solution.

10. The system of claim 9 wherein the hollow stylet is sized such that it is retained within the distal end of the catheter by friction.

11. The system of claim 9 wherein the treatment device is a coated stent.

12. The system of claim 11 wherein the stent is coated with one or more therapeutic agents selected from a group consisting of an antineoplastic agent, an antiproliferative agent, an antibiotic, an antithrombogenic agent, an anticoagulant, an antiplatelet agent, and an anti-inflammatory agent.

13. The system of claim 9 wherein the sleeve is sized such that it does not contact the treatment device.

14. The system of claim 9 further comprising:
a shipping and storage container, wherein the syringe fitting extends through a wall of the container.

15. The system of claim 14 further comprising:
a cap removably attached to the syringe fitting.

16. The system of claim 14 wherein at least one of the syringe fitting and the sleeve forms a unitary structure with a wall of the container.

17. A method of preparing a catheter for use, comprising:
providing a catheter system comprising a catheter and a catheter flushing device, the catheter flushing device including a syringe fitting, a hollow stylet, and a sleeve, the hollow stylet attached to and in fluid communication with the syringe fitting, the sleeve attached to the syringe fitting external to and coaxial with the hollow stylet, wherein the hollow stylet is received within a distal end of the catheter and the sleeve surrounds a distal portion of the catheter, the syringe fitting and hollow stylet serving as a flushing cannula for the catheter, the sleeve protecting the distal portion of the catheter from contact with a flushing solution;
attaching a syringe containing a flushing solution to the syringe fitting;
injecting the flushing solution into the catheter; and
removing the catheter flushing device from the catheter.

18. The method of claim 17 wherein the syringe fitting is a Luer fitting and wherein attaching a syringe to the syringe fitting comprises rotating a syringe onto the Luer fitting.

19. The method of claim 17 wherein the catheter system further comprises a shipping and storage container, and wherein the syringe fitting extends through a wall of the container.
